(19) United States
(12) Patent Application Publication
(10) Pub. No.: US 2025/0302628 A1
(43) Pub. Date: Oct. 2, 2025

(54) VERTEBRAL TETHERING WITH SOFT TISSUE GRAFT

(71) Applicant: BOARD OF SUPERVISORS OF LOUISIANA STATE UNIVERSITY AND AGRICULTURAL AND MECHANICAL COLLEGE, Baton Rouge, LA (US)

(72) Inventor: Rutledge Carter Clement, New Orleans, LA (US)

(21) Appl. No.: 19/090,767

(22) Filed: Mar. 26, 2025

Related U.S. Application Data
(60) Provisional application No. 63/570,195, filed on Mar. 26, 2024.

(51) Int. Cl. A61F 2/28; A61F 2/46

(52) U.S. Cl. CPC A61F 2/2846; A61F 2/4611; A61F 2002/285

(57) ABSTRACT

A vertebral tethering device is described herein comprising a tethering component. wherein the tethering component comprises a synthetic layer and a tissue graft layer, wherein the tethering component is affixed to vertebrae using a securing component.

This application claims the benefit of U.S. Application No. 63/570,195, filed Mar. 26, 2024.

### TECHNICAL FIELD

The disclosure herein involves vertebral tethering which is a surgical treatment for idiopathic scoliosis in growing children.

### BACKGROUND

Scoliosis is a condition in which a person's spine has an abnormal curve. The curve is usually S- or C-shaped over three dimensions. In some, the degree of curve is stable, while in others, it increases over time. Mild scoliosis does not typically cause problems, but more severe cases can affect breathing and movement. Pain is usually present in adults, and can worsen with age. As the condition progresses, it may impact a person's life and hence, can also be considered a disability.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### SUMMARY OF THE INVENTION

A vertebral tethering device is described herein comprising under an embodiment a tethering component, wherein the tethering component comprises a synthetic layer and a tissue graft layer, wherein the tethering component is affixed to vertebrae using a securing component.

The tethering component is affixed to a convex side of a scoliosis curve, under an embodiment.

The tissue graft layer faces the vertebrae, under an embodiment.

The tissue graft layer comprises an allograft, under an embodiment.

The allograft comprises an Achilles allograft, under an embodiment.

The tissue graft layer comprises an autograft, under an embodiment.

The autograft comprises a hamstring autograft, under an embodiment.

The securing component comprises a screw and soft tissue washer, under an embodiment.

A threaded element of the screw passes through the soft tissue washer from an upper side to a lower side, under an embodiment.

The screw is driven into a vertebra, under an embodiment.

The lower side of the soft tissue washer comprises conical spikes, under an embodiment.

The conical spikes tap into the synthetic layer under tension, under an embodiment.

The securing component comprises a suture anchor, under an embodiment.

A threaded anchor of the suture anchor is driven into a vertebra, under an embodiment.

Sutures are attached to the suture anchor through an eyelet, under an embodiment.

The sutures secure the tethering component to the vertebra, under an embodiment.

The securing component comprises a pedicle screw and tulip configuration, under an embodiment.

The pedicle screw is configured to drive through the tissue graft layer and into the vertebra, under an embodiment.

The pedicle screw is configured to threadably receive a tulip, wherein the secured tulip secures a washer between the tulip and the tissue graft layer, wherein the securing drives spikes of the washer into the tissue graft layer, under an embodiment.

The tulip comprises a tubular recess configured to receive the synthetic layer, under an embodiment.

The tulip is configured to threadably receive a set screw, wherein set screw secures the synthetic layer within the tubular recess, under an embodiment.

### BRIEF DESCRIPTION OF THE DRAWINGS

FIG. 1 shows a tether, under an embodiment.

FIG. 2 shows a profile view of a tether, under an embodiment.

FIG. 3 shows a tether affixed to the lateral side of vertebrae, under an embodiment.

FIG. 4 shows a screw and soft tissue washer for use in affixing a tether to vertebrae, under an embodiment.

FIG. 5 shows a stylized presentation of scoliosis featuring a double curve, under an embodiment.

FIG. 6 shows a suture anchor, under an embodiment.

FIG. 7 shows suture anchors driven into vertebrae, under an embodiment.

FIG. 8 shows a tether affixed to the lateral side of vertebrae, under an embodiment.

FIG. 9 shows a soft tissue graft attached to vertebral body using pedicle screw and tulip configuration, under an embodiment.

### DETAILED DESCRIPTION

Scoliosis is a sideways curve of the spine. Everyone has normal curves in the spine, and when looked at from behind, the spine appears straight. However, children and teens with scoliosis have an abnormal S-shaped or C-shaped curve of the spine. The curve can happen on either side of the spine and in different places in the spine. For most children and teens, the cause of scoliosis is idiopathic.

Treatments may be surgical or nonsurgical, and they also vary depending on the type of scoliosis a person has, and the age at which they develop it. For curves measuring less than 25 degrees, an orthopedist may recommend frequent monitoring to determine whether additional intervention becomes necessary. Young people with curves between 25 and 45 degrees may be candidates for treatment with bracing. While bracing does not correct the curve, it may stop its progression.

Patients with curves that continue to progress beyond 50 degrees, either with or without bracing, generally require surgical intervention. Spinal fusion is the most common type of surgery for scoliosis. Rods and screws are attached to the vertebrae to fuse the bones together, helping to straighten the spine. This procedure is only utilized for severe curves where all other treatment methods have failed.

Vertebral body tethering takes advantage of the spine's natural growth to correct sideways curvatures while allowing the spine to continue growing. Surgeons attach metal anchors to the vertebrae on the side of the spine that curves outward. A flexible cord or component, called a tether, is connected to these anchors and placed under tension. Over time, as the child continues to grow and their spine lengthens, the tether slows the growth on the curved side of the spine. This allows the other side of the spine to catch up. Over time, as a child grows, the spine grows straighter. The anchors and tether will remain attached to child's spine permanently unless problems develop.

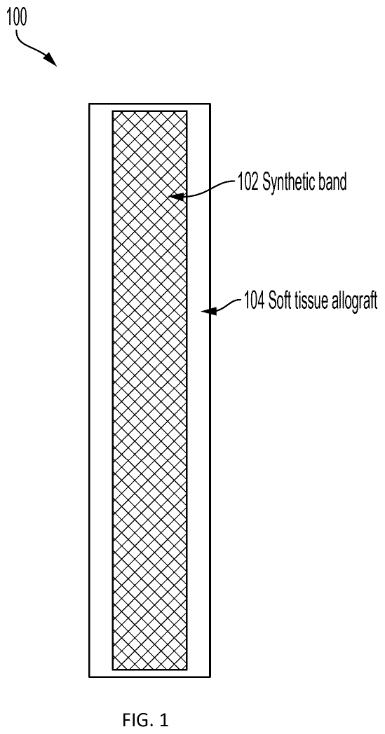

FIG. 1 shows a tether 100, under an embodiment. The tether 100 features a synthetic tether band 102 attached to a soft tissue allograft 104. The synthetic band comprises a robust cord. Under an embodiment, the cord is made of an ultra-high-molecular-weight polyethylene (UHMWPE) similar to other synthetic materials used to convey temporary strength in the body (e.g. Zimvie's “The Tether” or Arthrex's Internal Brace). A tissue allograft is tissue (i.e. bone, ligaments, heart valves) recovered from a human donor for transplantation into another person. Under an embodiment, the allograft shown in FIG. 1 may comprise an Achilles or other soft tissue allograft or autograft. Achilles allograft is particularly attractive as it is relatively strong and wide which would allow placement over a broad footprint on the lateral aspect of the vertebral bodies. Under an alternative embodiment, the tether may include soft tissue autograft as opposed to allograft. Autograft is a graft of tissue from one point to another of the same individual's body. If autograft were used, hamstring is attractive due to the length which may span the instrumented segment of the spine under an embodiment. Surgeons are familiar with the technique of hamstring harvest, and patients recover and function well after hamstring autograft use.

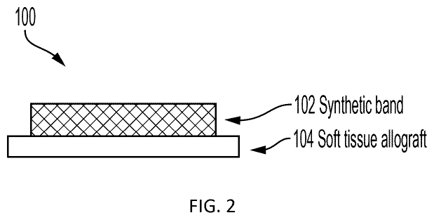

FIG. 2 provides a profile view of the vertebral tether 100. In operation, the soft tissue allograft 104 side of the tether faces the vertebrae.

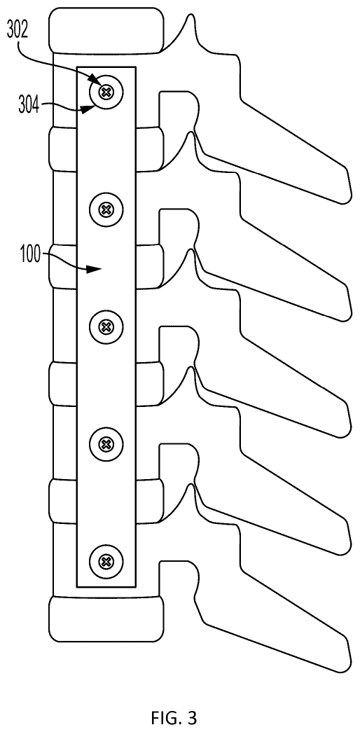

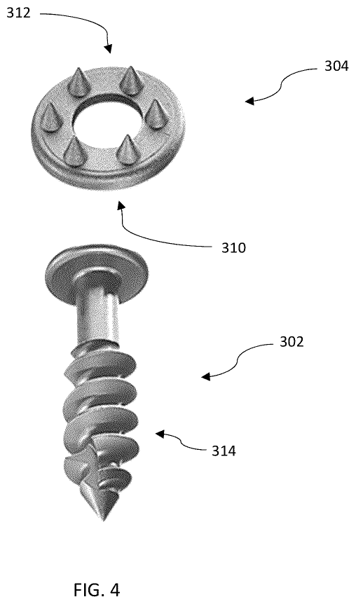

FIGS. 3 and 4 show an example of a tether 100 affixed to the lateral side of vertebrae using a series of screws 302 and soft tissue washers 304. The soft tissue washer is made of metal, it's named “soft tissue” washer because it holds down the soft tissue allograft. FIG. 4 illustrates the screw 302 and soft tissue washer 304 in disassembled configuration. The washer 304 features an upper side 310 and a lower side 312. A threaded end 314 of the screw 302 passes through the washer from upper side 310 to lower side 312. A surgeon then drives the screw through the synthetic band, through the allograft and into the vertebra. The lower side 312 of the soft tissue washer features conical spikes which tap into the synthetic band under tension.

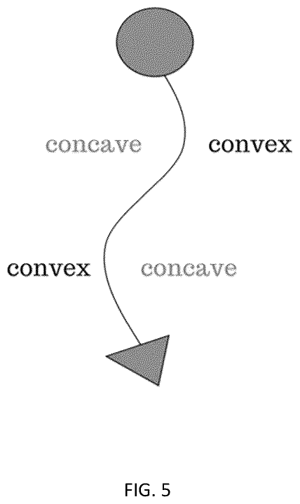

FIG. 5 provides a stylized presentation of scoliosis featuring a double (or S-shaped) curve. Both the upper and lower curves feature a convex and concave side. The vertebral tether described herein is attached to a convex side of either the upper or lower curve of both. Alternatively, tether 100 may be attached to the convex side of a C-shaped scoliosis curve. As seen in FIG. 3, a tether may be affixed to vertebrae using a screw and washer configuration. Additional means for affixing the vertebral tether are described below.

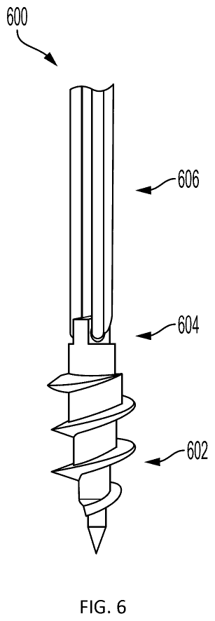

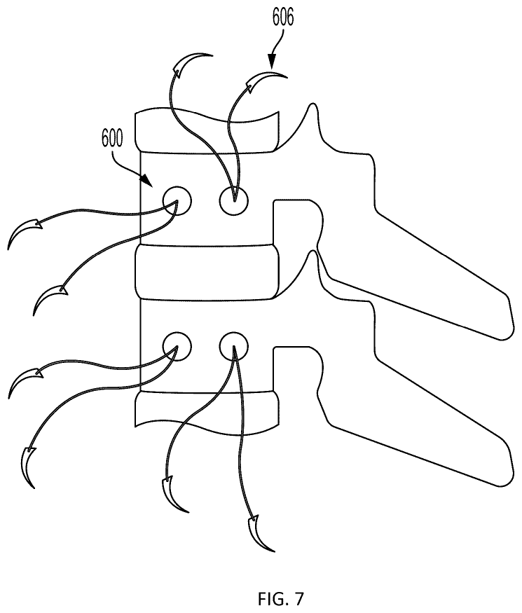

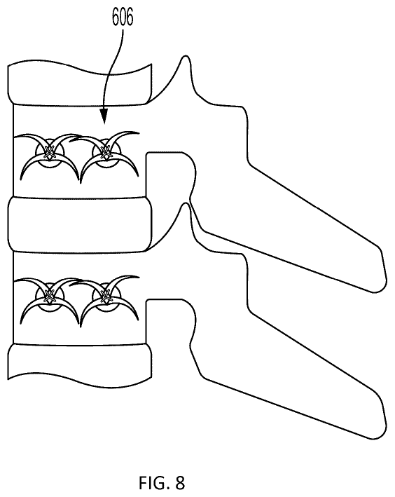

FIGS. 6, 7, and 8 show an example of fixing a tether to the lateral side of vertebrae using suture anchors 600. FIG. 6 illustrates a threaded anchor end 602 of a suture anchor 600. A proximal end of the anchor end comprises an eyelet 604. The suture 606 is attached to the anchor end through the eyelet. Under an embodiment, a surgeon drives two suture anchors 600 into each vertebra as seen in FIG. 7. Sutures 606 extend from each installed threaded anchor. Sutures 606 are then used to secure the tether to the vertebrae as seen in FIG. 8. Under an embodiment, these sutures compromise a heavy, braided, nonabsorbable material such as those typically used in suture anchors in sports medicine surgery.

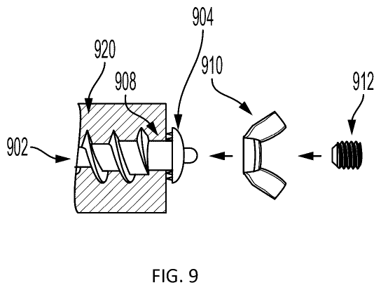

Under another embodiment, a modified pedicle screw with blunt tip and mechanism may be used for securing the allograft tether to the side of the vertebrae. Under an embodiment, the screw is modular in a way that secures the soft tissue allograft to the vertebra followed by attachment of a tulip that will secure the synthetic cord much like current vertebral body tethering systems, also allowing tensioning of the synthetic cord. As seen in FIG. 9, the shaft of a modified pedicle screw 902 is driven into the vertebral body. A modified soft tissue washer 904 resides over modular screw 902 to hold the soft tissue graft 920 against the vertebral body in cooperation with Tulip 910. The Tulip 910 threads onto the modular screw 902 and applies pressure to washer 904 so that conical spikes 908 of the washer tap into the tissue graft 920. A synthetic cord passes through the tubular recess of the Tulip. The set screw 912 threads onto the Tulip 910 and secures the position of a synthetic cord within the tubular recess of the Tulip. Under and alternative embodiment, the modified pedicle screw and tulip may comprise an integrally formed single implant. Under yet another alternative embodiment, the soft tissue graft and synthetic material could be affixed together by the screw or separately. In other words, the synthetic material may reside on the vertebral side of the soft tissue washer. In that case, the conical spikes 908 of the washer tap into the synthetic material. Under yet another embodiment, constructs only using soft tissue graft are possible.

Tethers may be removed after the patient has finished growing, but they are typically left in permanently unless they are causing a problem. Incorporating a soft tissue graft expands the indications to include skeletally mature patients.

Other mechanisms may be used for securing a soft tissue graft to vertebrae (with or without a synthetic cord). These include a form of staple, simple suturing to periosteum, pleura, or other tissue, and other customized screws, anchors, etc. designed for this purpose.

What is claimed is:

1. A vertebral tethering device comprising,
a tethering component, wherein the tethering component comprises a synthetic layer and a tissue graft layer, wherein the tethering component is affixed to vertebrae using a securing component.

2. The device of claim 1, wherein the tethering component is affixed to a convex side of a scoliosis curve.

3. The device of claim 2, wherein the tissue graft layer faces the vertebrae.

4. The device of claim 1, wherein the tissue graft layer comprises an allograft.

5. The device of claim 4, wherein the allograft comprises an Achilles allograft.

6. The device of claim 1, wherein the tissue graft layer comprises an autograft.

7. The device of claim 6, wherein the autograft comprises a hamstring autograft.

8. The device of claim 1, wherein the securing component comprises a screw and soft tissue washer.

9. The device of claim 8, wherein a threaded element of the screw passes through the soft tissue washer from an upper side to a lower side.

10. The device of claim 9, wherein the screw is driven into a vertebra.

11. The device of claim 10, wherein the lower side of the soft tissue washer comprises conical spikes.

12. The device of claim 11, wherein the conical spikes tap into the synthetic layer under tension.

13. The device of claim 1, wherein the securing component comprises a suture anchor.

14. The device of claim 13, wherein a threaded anchor of the suture anchor is driven into a vertebra.

15. The device of claim 14, wherein sutures are attached to the suture anchor through an eyelet.

16. The device of claim 15, wherein the sutures secure the tethering component to the vertebra.

17. The device of claim 1, wherein the securing component comprises a pedicle screw and tulip configuration.

18. The device of claim 17, wherein the pedicle screw is configured to drive through the tissue graft layer and into the vertebra.

19. The device of claim 18, wherein the pedicle screw is configured to threadably receive a tulip, wherein the secured tulip secures a washer between the tulip and the tissue graft layer, wherein the securing drives spikes of the washer into the tissue graft layer.

20. The device of claim 19, wherein the tulip comprises a tubular recess configured to receive the synthetic layer.

21. The device of claim 20. wherein the tulip is configured to threadably receive a set screw. wherein set screw secures the synthetic layer within the tubular recess.
